(19) United States
(12) Patent Application Publication
(10) Pub. No.: US 2002/0058788 A1
(43) Pub. Date: May 16, 2002

(54) Facile deprotection of Fmoc protected amino groups

(76) Inventor: James E. Sheppeck II, Wilmington, DE (US)

Correspondence Address: COOLEY GODWARD, LLP, 3000 EL CAMINO REAL, 5 PALO ALTO SQUARE, PALO ALTO, CA, 94306, US

(21) Appl. No.: 09/939,455

(22) Filed: Aug. 24, 2001

Related U.S. Application Data
(60) Provisional application No. 60/227,894, filed on Aug. 25, 2000.

(51) Int. Cl. C07K 1/00

(52) U.S. Cl. 530/335; 530/338

(57) ABSTRACT

The present invention provides a method for deprotecting a Fmoc protected amino group, said method comprising treating in a suitable medium the protected amino group with a base in the presence of a thiol compound to yield a deprotected amino group.

### CROSS REFERENCE TO RELATED APPLICATIONS

This application claims the benefit of U.S. Provisional Application No. 60/227,894, filed on Aug. 25, 2000.

### FIELD OF THE INVENTION

The present invention relates to a novel method of deprotecting a Fmoc protected amino group.

BACKGROUND OF THE INVENTION

Peptide synthesis involves building a complex molecule by undertaking a variety of chemical transformations. These chemical transformations can require protection of functional groups to render them unavailable as reactive centers. Protected functional groups are subsequently deprotected to facilitate derivatization of the desired molecule.

One of the functional groups that is routinely protected and deprotected during peptide synthesis is an amino group. Protection of the amino group involves establishing a covalent bond between the nitrogen atom of the amino group and a protecting group, while the deprotection step involves breaking this covalent bond to restore the amino group to its reactive free base form.

Different protecting groups have been used to protect an amino group to render it unreactive, and include the Boc, benzyloxycarbonyl (“CBZ”), allyloxycarbonyl (“Alloc”), β-(trimethylsilyl)-ethoxycarbonyl (“Teoc”) and Fmoc protecting groups. Procedures for protecting and deprotecting amino groups using these protecting groups are described in Protective Groups in Organic Synthesis, 3rd edition, T. W. Greene and P. G. M. Wuts, 1991. Molecules bound to solid supports or polymeric material and comprising amino groups protected with a Fmoc group are generally treated with piperidine or an equivalent base in DMF to facilitate the deprotection of the Fmoc protected amino group. This method works well and washing the deprotected solid bound molecule with an suitable solvent renders a clean product.

However, it is not convenient to deprotecting an amino group in solution phase using the above procedure. The difficulty arises from the fact that piperidine and similar bases along with solvents like DMF are high boiling and are difficult to remove from the unprotected amino product. Bases, such as piperidine, also tend to form a covalent adduct with dibenzofulvene which can also contaminate the final product. Thus, there is a need for a procedure to deprotect Fmoc protected amino groups to provide a clean product in solid as well as solution phase reactions.

### SUMMARY OF THE INVENTION

The invention relates to a method for deprotecting a Fmoc protected amino group.

One embodiment of the invention pertains to a method for deprotecting a Fmoc protected amino group comprising treating in an suitable medium the protected amino group with a base in the presence of a thiol compound to yield a deprotected amino group.

Another embodiment of the invention relates to a method for deprotecting a Fmoc protected amino group having the formula Fmoc-NR¹R², comprising treating in a suitable medium the protected amino group with a base in the presence of a thiol compound to yield a deprotected amino group having the Formula HNR¹R²; wherein R¹ and R² are moieties that bind to an amino group to form a stable compound.

Yet another embodiment of the invention pertains to a method for deprotecting a Fmoc protected amino group comprising treating in a suitable medium the protected amino group with a base in the presence of a thiol compound having the formula R³—SH to yield a deprotected amino group; wherein R³ is selected from the group consisting of aliphatic, aryl, heteroaryl and heterocycloalkyl moieties.

### DETAILED DESCRIPTION OF THE INVENTION

### DEFINITIONS

The following terms and phrases as used herein have the following meaning, unless indicated otherwise.

The term “base” is intended to mean an organic or inorganic substance with a pKa of greater than about 8. Exemplary bases include, by way of illustration and not limitation, DBU, pyridine, triethylamine, lutidine, diisopropylethylamine, piperidine, 1,5-diazabicyclo[4.3.0]non-5-ene and mixtures thereof.

“Suitable medium” as used herein, is meant to indicate a medium/solvent which is compatible with the reaction conditions and able to facilitate in the deprotection of the amino group. Representative examples of an suitable medium are THF, dioxane, toluene, DMF, dimethylsulfoxide, dimethyl acetamide, DCM, N-methyl pyrrolidinone, methanol, isopropanol, acetonitrile, hexanes, pyridine, benzene, a pure thiol and mixtures thereof. A list of suitable solvents can be found in Tetrahedron Letters 39:8451-54 (1998), which is incorporated herein by reference.

The term “thiol compound” is a compound comprising a thiol moiety attached to a aliphatic, aryl, heteroaryl or a heterocycloalkyl moiety. The thiol compound can also be written as R³—SH, wherein R³ is selected from the group consisting of aliphatic, aryl, heteroaryl and heterocycloalkyl moieties. Exemplary thiol compounds include by way of illustration, octane thiol, benzyl mercaptan, hexane thiol, cyclohexylmethane thiol, cyclohexane thiol, thiophenol, and thiol resins such as N-(2-mercaptp ethyl)aminomethyl polystyrene resin.

The term “aliphatic” includes “alkyl” and “cycloalkyl” moieties and is meant to indicate a saturated or partially unsaturated hydrocarbon moiety containing 1 to 14 carbon atoms. The aliphatic moiety is generally attached to at least one other atom, and can be a straight chain, branched or cyclic. An example of a partially unsaturated aliphatic moiety is an “alkelene” group which has at least one center of unsaturation, i.e., a double bond. Illustrative examples are butene, butadiene, propene, and pentene. A “cycloalkyl” moiety is a saturated or partially unsaturated 3 to 10 carbon monocyclic or bicyclic hydrocarbon moiety. The term straight chain alkyl moiety is meant to represent an unbranched hydrocarbon moiety of up to 8 carbon atoms such as n-pentyl. The aliphatic moiety can be substituted with 1-3 substituents selected from the group consisting of alkoxy, ═O, —N(C₁₋₄alkyl)₂, —OH, —C(O)—O—C₁₋₈alkyl, —S—C₁₋₄alkyl, cyano (—C≡N), —CF₃, halo, aryl, heteroaryl and —C₁₋₄alkylester.

The term “aryl” means an aromatic monocyclic, bicyclic, or a fused polycyclic hydrocarbon moiety containing the number of carbon atoms indicated, and which can be optionally substituted with 1-3 substituents selected from the group consisting of alkoxy, ═O, —N(C₁₋₄alkyl)₂, —OH, —C(O)—O—C₁₋₈alkyl, —S—C₁₋₄alkyl, cyano (—C≡N), —CF₃, halo, aryl, heteroaryl and C₁₋₄alkylester. Thus a C₆-C₁₄ aryl group includes phenyl, naphthyl, anthracenyl, etc. The term “heteroaryl” means aryl, as defined above, wherein one or more of the carbon atoms is replaced by a heteroatom chosen from N, O, and S. The heteroatoms can exist in any of their chemically allowed oxidation states, e.g., sulfur can exist as a sulfide, sulfoxide, or sulfone. Each heteroaryl ring comprises from 5 to 14 atoms and examples include thienyl, furyl, pyrrolyl, indolyl, pyrimidinyl, isoxazolyl, purinyl, imidazolyl, pyridyl, pyrazolyl, quinolyl, and pyrazinyl.

The term “heterocycloalkyl” moiety means a cycloalkyl, as defined above, where one or more of the carbon atoms of the cycloalkyl moiety are replaced by a heteroatom selected from the group consisting of N, NR, O, S(O), S(O)₂ and S, wherein R is —C₁₋₆alkyl or hydrogen. Illustrative heterocycloalkyl moieties include morpholinyl, indolinyl, piperidyl, pyrrolidinyl, pyrrolinyl, imidazolidinyl, quinuclidinyl and morpholinyl. The heterocycloalkyl moiety can be optionally substituted with 1-3 substituents selected from the group consisting of alkoxy, ═O, —N(C₁₋₄alkyl)₂, —OH, —C(O)—O—C₁₋₈alkyl, —S—C₁₋₄alkyl, cyano (—C≡N), —CF₃, halo, aryl, heteroaryl and —C₁₋₄alkylester.

The term “alkoxy” represents an oxygen atom attached to an alkyl moiety and comprises from 1-6 carbon atoms.

A “deprotected amino group” as used herein means a primary amine, a secondary amine or aniline.

The present invention provides a method for deprotecting a Fmoc protected amino group comprising treating in a suitable medium the protected amino group with a base in the presence of a thiol compound to yield a deprotected amino group.

The thiol compound can be bound to a solid support or it can be in solution. A preferred embodiment provides a method wherein the thiol compound is aliphatic. In another preferred embodiment the thiol group is attached to the rest of the thiol compound by a methylene group.

As noted above, examples of suitable media that can be used in the method of the invention include, by way of example and not limitation, THF, dioxane, toluene, DMF, dimethylsulfoxide, dimethyl acetamide, DCM, N-methyl pyrrolidinone, methanol, isopropanol, acetonitrile, hexanes, pyridine, benzene, a pure thiol and mixtures thereof. Preferred media include, THF, methanol, isopropanol, dioxane, toluene, acetonitrile, hexanes, pyridine, benzene and mixtures thereof. In one embodiment of the invention, the medium used in the method is an organic solvent with a boiling point below about 120° C.

Abbreviations

| | |
|---|---|
| AMC | 7-amino-4-methylcoumarin |
| Arg | arginine |
| Asp | aspartic acid |
| Asn | asparagine |
| Boc | t-butoxy carbonyl |
| Cit | citraline |
| Cys | cysteine |
| DBU | 1,8-diazabicyclo[5.4.o]undec-7-ene |
| DCM | dichloromethane |
| DMF | dimethylformamide |
| EtOH | ethanol |
| Et₂O | diethyl ether |
| Fmoc | 9-fluoromethoxy carbonyl |
| Gln | glycine |
| Glu | glutamic acid |
| Lys | lysine |
| O-t-BU | tert-butyl ether |
| Orn: | ornithine |
| Pbf | 2,2,4,6,7-pentamethyl di-hydrobenzofuran-5-sulfonyl |
| Ser | serine |
| t-Bu | tert-butyl |
| THF | tetrahydrofuran |
| Thr | threonine |
| Trp | tryptophan |
| Trt | trityl |
| Tyr | tyrosine |

### Experimental

The method of the present invention is further illustrated by the reaction Scheme I and synthetic procedures discussed below.

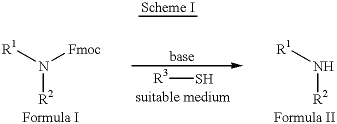

R¹ and R² are moieties that bind to an amino group to form a stable compound and R³—SH represents a compound comprising a thiol group. The R³ substituent can be selected from the group consisting of aliphatic, aryl, heteroaryl and heterocycloalkyl moieties.

“Moieties that bind to an amino group to form a stable compound” are also commonly referred to as “suitable amine substituent(s)” and represent substituents which are capable of forming a covalent bond with an amine group to form a compound of Formula I and/or II. Illustrative examples of suitable amine substituents are optionally substituted alkyls, optionally substituted aryls, optionally substituted heteroaryls and optionally substituted alkoxy groups. Such groups include, byway of illustration and not limitation, 2-hydroxy-1-hydroxymethyl-2-phenyl-ethylamino, 2-hydroxy-1-hydroxymethyl-3-methyl-pentylamino, 1-benzyl-2-hydroxy-ethylamino, 1-hydroxymethyl-3-methyl-butylamino, 4-amino-piperidine-1-carboxylic acid ethyl ester, 2-acetylamino-ethylamino, 2-diethylamino-ethylamino, 2-(2-hydroxy-ethoxy)-ethylamino, 3-diethylamino-propylamino, 3-hydroxy-propylamino, 6-hydroxy-hexylamino, 3-imidazol-1-yl-propylamino, 2-(4-sulfamoyl-phenyl)-ethylamino, 3-(4-methyl-piperazin-1-yl)-propylamino, 2-dimethylamino-1-methyl-ethylamino, 2-[bis-(2-hydroxy-ethyl)-amino]-ethylamino, 1-carbamoyl-2-phenyl-ethylamino, 2-dibutylamino-ethylamino, 5-hydroxy-4,4-dimethyl-pentylamino, 3-dimethylamino-2,2-dimethyl-propylamino, 2-(butyl-ethyl-amino)-ethylamino, 2-diisobutylamino-ethylamino, 2-hydroxy-butylamino, 3-hydroxy-2,2-dimethyl-propylamino, cyclohexylamino, (5-hydroxy-1,3,3-trimethyl-cyclohexylmethyl)-amino, 1,2,3,4-tetrahydro-naphthalen-1-ylamino, cyclooctylamino, 3-(2-oxo-pyrrolidin-1-yl)-propylamino, indan-1-ylamino, (tetrahydro-furan-2-ylmethyl)-amino, 2-(1h-indol-3-yl)-ethylamino, (benzo[1,3]dioxol-5-ylmethyl)-amino, 3-morpholin-4-yl-propylamino, 2-pyridin-2-yl-ethylamino, 2-hydroxy-1-methyl-2-phenyl-ethylamino, 1-methoxy-ethylamino, 1-methyl-3-phenyl-propylamino, 3-diethylamino-1-methyl-propylamino, benzylamino, 2-fluoro-benzylamino, 2-methoxy-benzylamino, 3-trifluoromethyl-benzylamino, 2-phenylamino-ethylamino, 2-methoxy-ethylamino, phenethylamino, 2-(2-methoxy-phenyl)-ethylamino, 2-(3,4-dimethoxy-phenyl)-ethylamino, 2-(4-chloro-phenyl)-ethylamino, 2-(4-methoxy-phenyl)-ethylamino, 2-(4-hydroxy-phenyl)-ethylamino, 3,3-diphenyl-propylamino, 2,5-dimethyl-benzylamino, 2-trifluoromethyl-benzylamino, butylamino, 1,2-diethyl-pyrazolidin-4-ylamino, 3-methoxy-propylamino, 2-diisopropylamino-ethylamino, 1-isopropyl-2-methyl-propylamino, 3-m-tolylamino-pentylamino, 3-butoxy-propylamino, 1-(4-fluoro-phenyl)-ethylamino, 1-methoxymethyl-propylamino, 2,3-dimethoxy-benzylamino, 2,4-dimethoxy-benzylamino, 2-(2-chloro-6-fluoro-benzylsulfanyl)-ethylamino, 2,6-dimethoxy-benzylamino, 3,5-dimethoxy-benzylamino, 2-phenoxy-ethylamino, 1-benzyl-pyrrolidin-3-ylamino, 2-(2,3-dimethoxy-phenyl)-ethylamino, 2-(2,5-dimethoxy-phenyl)-ethylamino, 2-(2-ethoxy-phenyl)-ethylamino, 2-(3,5-dimethoxy-phenyl)-ethylamino, 2-(4-ethoxy-phenyl)-ethylamino, 2-(4-trifluoromethoxy-phenyl)-ethylamino, 2-hydroxy-1,2-diphenyl-ethylamino, 2-hydroxy-1,2-diphenyl-ethylamino, 2-(2-hydroxymethyl-phenylsulfanyl)-benzylamino, 2-(3-fluoro-phenyl)-ethylamino, 2-(2-amino-phenyl)-benzylamino, 2-(2-fluoro-phenyl)-ethylamino, 4-amino-benzylamino, 2-(3,4-dimethoxy-phenyl)-ethylamino, 1 ,2-dihydroxy-2-(4-methylsulfanyl-phenyl)-ethylamino, 2-hydroxy-cyclohexylamino, and 3-(methyl-phenyl-amino)-propylamino. A comprehensive list of amines substituted with a substituted with suitable substituents can be found in the Aldrich chemicals catalog, which is incorporated herein by reference.

### Synthetic Procedure

Referring to Scheme I, a compound of Formula I (about 1 eq.; comprising a Fmoc protected amino group) is treated with a base (e.g., DBU; about 0.1 to 0.5 eq.) in a suitable medium (e.g., THF) in the presence of a compound comprising a thiol group (e.g., a thiol resin or octanethiol; about 5 to 15 eq.). The resulting reaction mixture is agitated at ambient temperature for up to about 60 hours. The reaction mixture is then concentrated to yield a compound of Formula II. Purification of the compound of Formula II, if necessary, can be accomplished by techniques known to one skilled in the art. Illustrative examples of such techniques are chromatography, recrystallization, trituration and the like.

Advantages of the present invention include the quantitative removal of the amino protecting Fmoc group using a catalytic amount of DBU base (0.1 to 0.25 eq) and 5-10 eq of a thiol or thiol resin. The resin scavenges the dibenzofulvene that comes from the Fmoc group, which can be removed by filtration, and the product can be freed from any residual amounts of DBU under reduced pressure or by trituration. This is a considerable improvement over the classical deprotection route described above where the Fmoc group is treated with piperidine or an equivalent base in DMF, to facilitate the deprotection of the Fmoc protected amino group.

### EXAMPLES

The following representative examples further illustrate the method of the present invention. The compounds can be treated with a base in a suitable medium to form the corresponding unprotected amino group containing compounds of Formula II, using the method of the present invention.

### Example 1

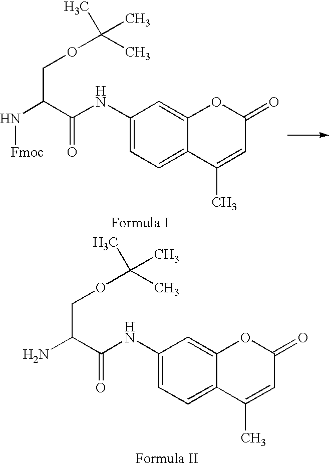

H-Lys(Boc)-AMC (Formula II)

Fmoc-Lys(Boc)-AMC (Formula I) (15.9 g, 25.5 mmol), 1-octanethiol (44.2 mL, 255 mmol), DBU (114.3 μL, 0.77 mmol, 3 mol %). Reaction time: 3.25 h. Yield 10.3 g (quant.).

MS 404 (M+H⁺).

¹H NMR (CD₃OD) δ 7.80 (s, 1H), 7.67 (d, 1H), 7.47 (d, 1H), 6.19 (s, 1H), 3.42 (m, 1H), 2.99 (m, 2H), 2.41 (s, 3H), 1.72 (m, 2H), 1.60 (m, 2H), 1.44 (m, 2H), 1.35 (s, 9H).

### Example 2

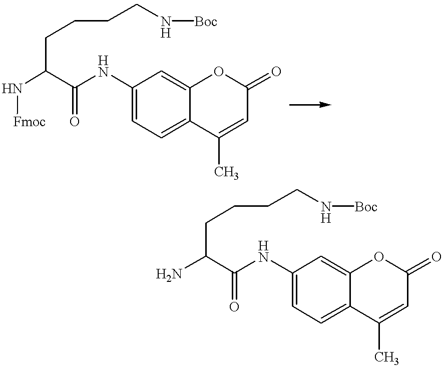

H-Orn(Boc)-AMC (Formula II)

Fmoc-Orn(Boc)-AMC (Formula I) (25.5 g, 41.7 mmol), 1-octanethiol (72 mL, 417 mmol), DBU (623 μL, 4.17 mmol, 10 mol %). Reaction time: 14 h. This compound was obtained as an oil which then was taken up in 50 mL EtOH, 1.1 eq of glacial acetic acid was added, the solution was concentrated in vacuo, taken up in DCM, and triturated with Et₂O at −20° C. Yield of acetate salt: 17.27 (92%).

MS 389 (M⁺)

### Example 3

H-Cit-AMC (Formula II)

Fmoc-Cit-AMC (Formula I) (16.4 g, 30 mmol), 1-octanethiol (51.2 mL, 295 mmol), DBU (441 μL, 2.94 mmol, 10 mol %). Reaction time: 14 h: Yield: 8.35 (85%).

MS 333 (M+H⁺)

H-Ser(t-Bu)-AMC (Formula II)

Fmoc-Ser(t-Bu)-AMC (Formula I) (19.3 g, 35.7 mmol), 1-octanethiol (62.0 mL, 357 mmol), DBU (533 μL, 3.57 mmol, 10 mol %). Reaction time: 3 h. Yield 8.0 g (70.0%).

MS 319 (M+H⁺)

¹H NMR (CD₃OD) δ 7.78 (s, 1H), 7.65 (d, 1H), 7.45 (d, 1H), 6.18 (s, 1H), 3.56 (m, 2H), 3.26 (m, 1H), 2.40 (s, 3H), 1.13 (s, 9H).

H-Tyr(t-Bu)-AMC (Formula II)

Fmoc-Tyr(t-Bu)-AMC (Formula I) (41.4 g, 67.1 mmol), 1-octanethiol (116.5 mL, 671 mmol), DBU (3 mL, 20.0 mmol, 30 mol %). Reaction time: 24 h. Yield 15.8 g (59.8%).

MS 395 (M+H⁺)

¹H NMR (CD₃OD) δ 7.65-7.34 (m, 3H), 7.10 (d, 2H), 6.84 (d, 2H), 6.18 (s, 1H), 3.69 (t, 1H), 2.94 (m, 2H), 2.39 (s, 3H), 1.92 (s, 9H).

H-Asp(O-t-Bu)-AMC (Formula II)

Fmoc-Asp(O-t-Bu)-AMC (Formula I) (34.5 g, 60.8 mmol), 1-octanethiol (105.4 mL, 608 mmol), DBU (1.8 mL, 12.0 mmol, 20 mol %). Reaction time: 28 h. Yield 16.8 g (79.7%).

MS 347 (M+H⁺)

¹H NMR (1:1 CDCl₃/CD₃OD) δ 7.52 (s, 1H), 7.35 (d, 1H), 7.24 (d, 1H), 5.94 (s, 1H), 3.55 (t, 1H), 2.49 (m, 2H), 2.18 (s, 3H), 1.17 (s, 9H).

H-Glu(O-t-Bu)-AMC (Formula II)

Fmoc-Glu(O-t-Bu)-AMC (Formula I) (39.7 g, 68.1 mmol), 1-octanethiol (118.1 mL, 681 mmol), DBU (1.02 mL, 6.81 mmol, 10 mol %). Reaction time: 48 h. Yield 22.1 g (90.2%).

MS 361 (M+H⁺)

¹H NMR (CD₂Cl₂) δ 9.86 (br s, 1H), 7.76 (s, 1H), 7.59 (d, 1H), 7.51 (d, 1H), 6.18 (s, 1H), 3.53 (m, 1H), 2.43 (s, 3H), 2.04 (m, 2H), 1.61 (m, 2H), 1.47 (s, 9H).

H-Thr(t-Bu)-AMC (Formula II)

Fmoc-Thr(t-Bu)-AMC (Formula I) (20.8 g, 37.6 mmol), 1-octanethiol (65.2 mL, 376 mmol), DBU (562 μL, 3.76 mmol, 10 mol %). Reaction time: 5 h. Yield 6.0 g (74.2%).

MS 333 (M+H⁺)

¹H NMR (CD₃OD) δ 7.81 (s, 1H), 7.69 (d, 1H), 7.46 (d, 1H), 6.20 (s, 1H), 4.05 (m, 1H), 3.21 (m, 1H), 2.42 (s, 3H), 1.18 (d, 3H), 1.13 (s, 9H).

H-Trp(Boc)-AMC (Formula II)

Fmoc-Trp(Boc)-AMC (Formula I) (10.7 g, 15.7 mmol), 1-octanethiol (27.3 mL, 157 mmol), DBU (235 μL, 1.57 mmol, 10 mol %). Reaction time: 19 h. Yield 5.6 g (77.8%).

MS 462 (M+H⁺)

¹H NMR (CD₃OD) δ 8.04-7.12 (m, 8H), 6.18 (s, 1H), 3.74 (t, 1H), 3.09 (m, 2H), 2.40 (s, 3H), 1.55 (s, 9H).

H-Arg(Pbf)-AMC (Formula II)

Fmoc-Arg(Pbf)-AMC (Formula I) (19.6 g, 24.3 mmol), 1-octanethiol (42.2 mL, 243 mmol), DBU (728 μL, 4.87 mmol, 20 mol %). Reaction time: 19 h. Yield 8.63 g (60.8%).

MS 584 (M+H⁺)

¹H NMR (CD₃OD) δ 7.18 (m, 1H), 7.66 (m, 1H), 7.47 (m, 1H), 6.19 (s, 1H), 3.53 (m, 2H), 3.43 (t, 1H), 3.15 (in, 2H), 2.91 (s, 2H), 2.49 (s, 3H), 2.43 (s, 3H), 2.41 (s, 3H), 1.99 (s, 3H), 1.38 (s, 6H), 1.25 (m, 2H).

H-Asn(Trt)-AMC (Formula II)

Fmoc-Asn(Trt)-AMC (Formula I) (47.2 g, 62.5 mmol), 1-octanethiol (108.5 mL, 625 mmol), DBU (935 μL, 6.25 mmol, 10 mol %). Reaction time: 22.5 h. Yield 27.0 g (81.1%).

MS 532 (M+H⁺)

¹H NMR (CD₂Cl₂) δ 10.0 (br s, 1H), 7.82-7.19 (m, 18H), 6.18 (s, 1H), 3.72 (br s, 1H), 3.36 (m, 1H), 2.84 (m, 2H), 2.43 (s, 3H).

H-Gln(Trt)-AMC (Formula II)

Fmoc-Gln(Trt)-AMC (Formula I) (48.7 g, 63.4 mmol), 1-octanethiol (110.0 mL, 634 mmol), DBU (948 μL, 6.34 mmol, 10 mol %). Reaction time: 21.5 h. Yield 28.9 g (83.6%).

MS 546 (M+H⁺)

¹H NMR (CD₂Cl₂) δ 9.85 (br s, 1H), 7.76 (s, 1H), 7.57 (d, 1H), 7.41 (d, 1H), 7.36-7.22 (m, 15H), 6.17 (s, 1H), 3.41 (m, 1H), 2.52 (t, 2H), 2.42 (s, 3H), 2.03 (m, 2H).

H-Cys(Trt)-AMC (Formula II)

Fmoc-Cys(Trt)-AMC (Formula I) (56.7 g, 76.4 mmol), 1-octanethiol (132.5 mL, 764 mmol), DBU (1.14 mL, 7.64 mmol, 10 mol %). Reaction time: 30 h. Yield 33.8 g (88.2%).

MS 521 (M+H⁺)

¹H NMR (CD₂Cl₂) δ 9.64 (br s, 1H), 7.69-7.22 (m 18H), 6.16 (s, 1H), 3.24 (m, 1H), 2.75 (m, 2H), 2.41 (s, 3H).

Each of the patent applications, patents, publications, and other published documents mentioned or referred to in this specification is herein incorporated by reference in its entirety, to the same extent as if each individual patent application, patent, publication, and other published document was specifically and individually indicated to be incorporated by reference.

While the present invention has been described with reference to the specific embodiments thereof, it should be understood by those skilled in the art that various changes may be made and equivalents may be substituted without departing from the true spirit and scope of the invention and the appended claims. In addition, many modifications may be made to adapt a particular situation, material, composition of matter, method, process step or steps, to the objective, spirit and scope of the present invention. All such modifications are intended to be within the scope of the claims appended hereto.

What is claimed is:

1. A method for deprotecting a Fmoc protected amino group, said method comprising treating in a suitable medium the protected amino group with a base in the presence of a thiol compound to yield a deprotected amino group.

2. The method of claim 1 wherein said base is selected from the group consisting of 1,8-diazabicyclo[5.4.o]undec-7-ene, pyridine, triethylamine, lutidine, diisopropylethylamine, piperidine, 1,5-diazabicyclo[4.3.0]non-5-ene and mixtures thereof.

3. The method of claim 1 wherein the thiol compound is bound to a solid support or is in solution.

4. The method of claim 3 wherein the thiol compound is bound to a solid support.

5. The method of claim 3 wherein the thiol compound is in solution.

6. The method of claim 1 wherein the thiol compound is aliphatic.

7. The method of claim 5 wherein the thiol compound comprises a thiol group attached to said compound by a methylene group.

8. The method of claim 1 wherein the thiol compound is selected from the group consisting of octane thiol, benzyl mercaptan, N-(2-mercaptp ethyl)aminomethyl polystyrene resin, hexane thiol, cyclohexylmethane thiol, cyclohexane thiol and thiophenol.

9. The method of claim 8 wherein the medium is selected from THF, methanol, isopropanol, dioxane, toluene, acetonitrile, hexanes, pyridine, benzene or mixtures thereof.

10. The method of claim 1 wherein the medium is selected from the group consisting of tetrahydrofuran, dioxane, toluene, dimethylformamide, dimethylsulfoxide, dimethyl acetamide, dichloromethane, N-methyl pyrrolidinone, methanol, isopropanol, acetonitrile, hexanes, pyridine, benzene, a pure thiol and mixtures thereof.

11. The method of claim 1 wherein the medium has a boiling point below about 120° C.

12. The method of claim 1 wherein the Fmoc protected amino group is used in an amount equal to about 1 equivalents, the base is used in an amount equal to about 0.1 to 0.5 equivalents and the thiol compound is used in an amount equal to about 5 to 15 equivalents.

13. The method of claim 12 wherein the base is used in an amount equal to about 0.1 to 0.25 equivalents and the thiol compound is used in an amount equal to about 5 to 10 equivalents.

14. The method of claim 1 wherein the base is 1,8-diazabicyclo[5.4.o]undec-7-ene and the thiol compound is 1-octanetiol.

15. A method for deprotecting a Fmoc protected amino group having the formula Fmoc-NR¹R², said method comprising treating in a suitable medium the protected amino group with a base in the presence of a thiol compound to yield a deprotected amino group having the Formula HNR¹R²; wherein R¹ and R² are moieties that bind to an amino group to form a stable compound.

16. The method of claim 15 wherein said base is selected from the group consisting of 1,8-diazabicyclo[5.4.o]undec-7-ene, pyridine, triethylamine, lutidine, diisopropylethylamine, piperidine, 1,5-diazabicyclo[4.3.0]non-5-ene and mixtures thereof.

17. The method of claim 15 wherein the thiol compound is bound to a solid support or is in solution.

18. The method of claim 17 wherein the thiol compound is selected from the group consisting of octane thiol, benzyl mercaptan, N-(2-mercaptp ethyl)aminomethyl polystyrene resin, hexane thiol, cyclohexylmethane thiol, cyclohexane thiol and thiophenol.

19. The method of claim 15 wherein the medium is selected from the group consisting of tetrahydrofuran, dioxane, toluene, dimethylformamide, dimethylsulfoxide, dimethyl acetamide, dichloromethane, N-methyl pyrrolidinone, methanol, isopropanol, acetonitrile, hexanes, pyridine, benzene, a pure thiol and mixtures thereof.

20. The method of claim 15 wherein R¹ and R² are selected from the group consisting of optionally substituted alkyls, optionally substituted aryls, optionally substituted heteroaryls and optionally substituted alkoxy groups.

21. The method of claim 20 wherein R¹ and R² are selected from the group consisting of 2-hydroxy-1-hydroxymethyl-2-phenyl-ethylamino, 2-hydroxy-1-hydroxymethyl-3-methyl-pentylamino, 1-benzyl-2-hydroxy-ethylamino, 1-hydroxymethyl-3-methyl-butylamino, 4-amino-piperidine-1-carboxylic acid ethyl ester, 2-acetylamino-ethylamino, 2-diethylamino-ethylamino, 2-(2-hydroxy-ethoxy)-ethylamino, 3-diethylamino-propylamino, 3-hydroxy-propylamino, 6-hydroxy-hexylamino, 3-imidazol-1-yl-propylamino, 2-(4-sulfamoyl-phenyl)-ethylamino, 3-(4-methyl-piperazin-1-yl)-propylamino, 2-dimethylamino-1-methyl-ethylamino, 2-[bis-(2-hydroxy-ethyl)-amino]-ethylamino, 1-carbamoyl-2-phenyl-ethylamino, 2-dibutylamino-ethylamino, 5-hydroxy-4,4-dimethyl-pentylamino, 3-dimethylamino-2,2-dimethyl-propylamino, 2-(butyl-ethyl-amino)-ethylamino, 2-diisobutylamino-ethylamino, 2-hydroxy-butylamino, 3-hydroxy-2,2-dimethyl-propylamino, cyclohexylamino, (5-hydroxy-1,3,3-trimethyl-cyclohexylmethyl)-amino, 1,2,3,4-tetrahydro-naphthalen-1-ylamino, cyclooctylamino, 3-(2-oxo-pyrrolidin-1-yl)-propylamino, indan-1-ylamino, (tetrahydro-furan-2-ylmethyl)-amino, 2-(1h-indol-3-yl)-ethylamino, (benzo[1,3]dioxol-5-ylmethyl)-amino, 3-morpholin-4-yl-propylamino, 2-pyridin-2-yl-ethylamino, 2-hydroxy-1-methyl-2-phenyl-ethylamino, 1-methoxy-ethylamino, 1-methyl-3-phenyl-propylamino, 3-diethylamino-1-methyl-propylamino, benzylamino, 2-fluoro-benzylamino, 2-methoxy-benzylamino, 3-trifluoromethyl-benzylamino, 2-phenylamino-ethylamino, 2-methoxy-ethylamino, phenethylamino, 2-(2-methoxy-phenyl)-ethylamino, 2-(3,4-dimethoxy-phenyl)-ethylamino, 2-(4-chloro-phenyl)-ethylamino, 2-(4-methoxy-phenyl)-ethylamino, 2-(4-hydroxy-phenyl)-ethylamino, 3,3-diphenyl-propylamino, 2,5-dimethyl-benzylamino, 2-trifluoromethyl-benzylamino, butylamino, 1,2-diethyl-pyrazolidin-4-ylamino, 3-methoxy-propylamino, 2-diisopropylamino-ethylamino, 1-isopropyl-2-methyl-propylamino, 3-m-tolylamino-pentylamino, 3-butoxy-propylamino, 1-(4-fluoro-phenyl)-ethylamino, 1-methoxymethyl-propylamino, 2,3-dimethoxy-benzylamino, 2,4-dimethoxy-benzylamino, 2-(2-chloro-6-fluoro-benzylsulfanyl)-ethylamino, 2,6-dimethoxy-benzyl amino, 3, 5-dimethoxy-benzylamino, 2-phenoxy-ethylamino, 1-benzyl-pyrrolidin-3-ylamino, 2-(2,3-dimethoxy-phenyl)-ethylamino, 2-(2,5-dimethoxy-phenyl)-ethylamino, 2-(2-ethoxy-phenyl)-ethylamino, 2-(3,5-dimethoxy-phenyl)-ethylamino, 2-(4-ethoxy-phenyl)-ethylamino, 2-(4-trifluoromethoxy-phenyl)-ethylamino, 2-hydroxy-1,2-diphenyl-ethylamino, 2-hydroxy-1,2-diphenyl-ethylamino, 2-(2-hydroxymethyl-phenylsulfanyl)-benzylamino, 2-(3-fluoro-phenyl)-ethylamino, 2-(2-amino-phenyl)-benzylamino, 2-(2-fluoro-phenyl)-ethylamino, 4-amino-benzylamino, 2-(3,4-dimethoxy-phenyl)-ethylamino, 1,2-dihydroxy-2-(4-methylsulfanyl-phenyl)-ethylamino, 2-hydroxy-cyclohexylamino, and 3-(methyl-phenyl-amino)-propylamino.

22. A method for deprotecting a Fmoc protected amino group, said method comprising treating in a suitable medium the protected amino group with a base in the presence of a thiol compound having the formula R³—SH to yield a deprotected amino group; where R³ is selected from the group consisting of aliphatic, aryl, heteroaryl and heterocycloalkyl moieties.

23. The method of claim 22 wherein said base is selected from the group consisting of 1,8-diazabicyclo[5.4.o]undec-7-ene, pyridine, triethylamine, lutidine, diisopropylethylamine, piperidine, 1,5-diazabicyclo[4.3.0]non-5-ene and mixtures thereof.

24. The method of claim 22 wherein the thiol compound is bound to a solid support or is in solution.

25. The method of claim 24 wherein the thiol compound is bound to a solid support.

26. The method of claim 24 wherein the thiol compound is in solution.

27. The method of claim 22 wherein R³ is an aliphatic group.

28. The method of claim 27 wherein the thiol compound comprises a thiol group attached to said compound by a methylene group.

29. The method of claim 22 wherein R³ is an aryl group.

30. The method of claim 22 wherein R³ is a heteroaryl group.

31. The method of claim 22 wherein R³ is a heterocycloalkyl group.

32. The method of claim 22 wherein the thiol compound is selected from the group consisting of octane thiol, benzyl mercaptan, N-(2-mercaptp ethyl)aminomethyl polystyrene resin, hexane thiol, cyclohexylmethane thiol, cyclohexane thiol and thiophenol.

33. The method of claim 22 wherein the medium is selected from the group consisting of tetrahydrofuran, dioxane, toluene, dimethylformamide, dimethylsulfoxide, dimethyl acetamide, dichloromethane, N-methyl pyrrolidinone, methanol, isopropanol, acetonitrile, hexanes, pyridine, benzene, a pure thiol and mixtures thereof.
